(19) United States
(12) Patent Application Publication
(10) Pub. No.: US 2026/0350790 A1
(43) Pub. Date: Nov. 5, 2026

(54) ARTIFICIAL INTELLIGENCE SYSTEM FOR TUMOR DIAGNOSTICS OF CLINICAL MAGNETIC RESONANCE IMAGING DATASETS

(71) Applicant: The Research Foundation for the State University of New York, Albany, NY (US)

(72) Inventors: Janet PALUH, Albany, NY (US); Ayan CHATTERJEE, Boston, MA (US); Amitava MUKHERJEE, Kolkata (IN)

(21) Appl. No.: 19/483,951

(22) PCT Filed: May 13, 2024
(86) PCT No.: PCT/US2024/029027

§ 371 Date: Nov. 12, 2025

Related U.S. Application Data
(60) Provisional application No. 63/465,719, filed on May 11, 2023.
(60) Provisional application No. 63/617,148, filed on Jan. 3, 2024.

(51) Int. Cl. G06T 7/00; G06N 3/0464

(52) U.S. Cl. CPC G06T 7/0012; G06N 3/0464; G06T 2207/10088; G06T 2207/20081; G06T 2207/30016; G06T 2207/30096

(57) ABSTRACT

A deep-learning artificial intelligence (AI) framework that applies neuromorphic analysis to complex clinical MRI DICOM datasets to enable typing of brain tumors that are glioblastomas or of metastatic origin with high accuracy. The data processing is optimized to address MRI modality class imbalance, remove lack of comparability, and aid generalizability to create a computationally light, portable, highly accurate and reliable diagnosis platform. The present AI capabilities to continue learning from complex and diverse clinical MRI datasets brings MRI robustness in biomedical neurosurgical treatment pre-planning, with applications in other brain pathologies and expanded use in artificial generalizable intelligence.

### CROSS-REFERENCE TO RELATED APPLICATIONS

This application claims the benefit of U.S. Provisional Patent Application Nos. 63/465,719, filed May 11, 2023, and 63/617,148, filed Jan. 3, 2024, the entireties of which are hereby incorporated herein by this reference.

### BACKGROUND OF THE INVENTION

### 1. Field of the Invention

The present invention generally relates to artificial intelligence systems and medical diagnostics. More particularly, the present invention relates to a system and method for diagnosing brain tumors from clinical magnetic resonance imaging (MRI) data images though the use of artificial intelligence models.

### 2. Description of the Related Art

In general, Brain tumor neoplasms arise via cerebral central nervous system (CNS) primary origin or by metastasis from alternate sites of pathology, including tumors of the lung, breast and melanomas, and rank globally as the 10th leading cause of death in US. The average age adjusted incidence rate (AAIR, 2012-2016) of all CNS tumors is 23.4 per 100,000. Brain metastases are most common versus primary intracranial tumors and approximately 30% of brain and CNS tumors are malignant, with tumor types and grades varying with age and demographics. Of malignant primary brain tumors, gliomas dominate at ~75% of tumors and include glioblastoma multiforme (GBM, 14.6%) that are most aggressive. The highly invasive nature of GBMs require radical tumor resection in treating the disease, with poor survival rates of <5% of patients surviving 5 years after diagnosis.

Neurosurgical strategies of treatment differ between cerebral metastases or glioblastomas, yet differentiating tumor types via magnetic resonance imaging (MRI) radiology remains challenging and impacts medical workup and treatment. A reliance on a range of information is required such as patient history of systemic malignancy and tumor features such as cerebral location near grey or white matter, multiplicity of cerebral sites, MR tumor morphology, and MR perfusion for tumor vascularity to provide predictive insight, although none of these factors are absolute. Intraoperative biopsies for genetic analysis further allow biomarker typing of tumors which assists in treatment. Despite these challenges, MRI remains a primary tool for initial guidance of tumor pathology.

One of the first demonstrated uses of MRI was its ability to resolve physiologic detail of normal versus pathological tumors in a rat model. A decade later, clinical use of 1.5 and 2.0 Tesla MRI on human patients was implemented and recent FDA approval of 7.0 Tesla MRI brings possibilities for very significant levels of physiological detail. The reliance on trained medical experts to evaluate MRI data is expected to become more strained with technology advances that require additional training, adding to time demands on radiologists to review and turnaround diagnostics on images. Given the increasing demand on radiologist and neurosurgeon clinicians in patient care, the early implementation of artificial intelligence for deep learning analysis of clinical data for MRI has become imperative.

Currently development of cutting-edge AI platforms has focused on detection of tumor pathology, such as the deep learning SCENIC algorithm developed by this group and tumor segmentation for detecting tumor edges. To what extent pre-processing of clinical data or synthesized data applied to AI development, data training and validation introduces noise or results in loss of data remains relatively unexplored. For example, complex operations such as skull stripping and MR bias field correction are sometimes applied. It is important to achieve comparable performance on medical data for generalizability and high transferability to clinical settings.

One previously developed system was as a deep learning neoplasm identification platform, with 98.3% classification accuracy on the publicly available BraTS Dataset. Although accurate for identifying tumor presence across MRI modalities, the previous design did not permit differentiation of glioblastomas or metastasized tumors (herein referred to as GBM or MET). Although clinical data is abundant, acquisition agreements with hospitals are required to ensure patient privacy through Institutional Review Board (IRB) agreements and clinical expertise and additional diagnostics must be present to verify tumor type.

It is thus to addressing the problems in existing AI models that can assist with prediction and treatment of brain tumors, as well as other cancer diagnoses, that the present invention is primarily directed.

### BRIEF SUMMARY OF THE INVENTION

Briefly described, the present system and method is a deep learning platform, called SIENNA, developed with access to a non-processed de-identified clinical DICOM dataset of clinically identified brain tumors, and non-tumor scans. Described herein are key challenges and strategies in working with clinical data that employ cutting-edge algorithms and tools to guide optimizations. This includes Synthetic Minority Over-Sampling Technique (SMOTE) to address class imbalance, “Hyperas” for optimizing hyper-parameters, histogram equalization for scan standardization, and adversarial training to ensure model robustness against different types of adversarial attacks. Capitalizing on the distinctive attributes of the convolutional neural network (CNN) architecture and its capacity to extract intricate hierarchical features from data, the clinical data optimized SIENNA model exhibits robust tissue pathology typing, demonstrated by discrimination of GBM versus MET tumor pathologies. The versatility of SIENNA in handling both raw and processed clinical data renders it an indispensable resource for enhancing clinical treatment workflow speed and efficacy to promote superior patient outcomes.

In one embodiment, the invention includes a system for determining the presence of cancerous tumors within diagnostic images of a human body. There is a scanning device generating one or more diagnostic images of a human body, such as an MRI device. The scanning device selectively transmits the one or more diagnostic images to other computer devices either through a wired or wireless connection, and there is a receiving computer device configured to process the received images. The computer device is configured to receive the one or more diagnostic images from the scanning device, create a plurality of tiles for each received image, compute a histogram for each tile, redistribute an intensity for each tile, create a cumulative distribution function scaling for each tile, conduct a bilinear interpolation to thereby stitch together the processed tiles, combine the processed tiles into in a final equalized image, and analyze the final equalized image to determine a presence of cancerous tumors within the diagnostic images.

In an embodiment, the invention also includes a computer-implemented method for determining the presence of cancerous tumors within diagnostic images of a human body. The method can be implemented by performing the steps of generating one or more diagnostic images of a human body at a scanning device, selectively transmitting the one or more diagnostic images from the scanning device to other computer devices, receiving the one or more diagnostic images from the scanning device, creating a plurality of tiles for each received image, computing a histogram for each tile, redistributing an intensity for each tile, creating a cumulative distribution function scaling for each tile, conducting a bilinear interpolation to thereby stitch together the processed tiles, combining the processed tiles into in a final equalized image, and then analyzing the final equalized image to determine a presence of cancerous tumors within the diagnostic images.

The present invention therefore provides an advantage in the diagnosis of tumors. Further, the present invention is industrially applicable as it provides a medical diagnostic system to aid healthcare providers in giving an accurate medical diagnosis and providing effective treatment. Other advantages, features, and benefits of the present invention will be apparent to one of skill in the art after review of the present application.

### BRIEF DESCRIPTION OF THE DRAWINGS

FIG. 1 is a diagram of one embodiment of the present system utilizing an MRI machine to provide diagnostic images of a human brain.

FIG. 2A is an overview and representation of MRI clinical dataset illustrating: a. Clinical workflow from MRI scans to SIENNA output interpretation by neurosurgeons, including collection, preprocessing, input, and output interpretation of data

FIG. 2B is a series of diagnostic images that are examples of de-identified raw MRI scans deemed noisy and unfit for training.

FIG. 2C is a series of MRI scans across different modalities present in the clinical dataset are shown along with representative images from GBM and MET.

FIG. 3A is a series of images and graphs illustrating histogram equalization.

FIG. 3B is a series of MRI images adversarial examples used in the training process.

FIG. 3C is a table of Hyperas describing the various parameters and their respective plausible ranges of values provided for hyperparameter tuning.

FIG. 3D is a chart of the comparison of histogram equalization algorithms tested and evaluated using the SSIM index.

FIG. 4A illustrates the SIENNA CNN Multilayer CNN

FIG. 4B is a table summarizing the layers and output of the CNN architecture—includes the number of layers, output shape, and the number of parameters.

FIG. 4C is a diagram illustrating the SIENNA data processing pipelines.

FIG. 5A is a series of MRI images highlighting the most frequently correctly and incorrectly classified samples.

FIG. 5B is a table of the performance evaluation on clinical data of the evaluation metrics estimated as an average of 100 runs of SIENNA.

FIG. 5C is a table of the performance evaluation of SIENNA for binary classification tasks on the extensively preprocessed BraTS 2020 dataset.

FIG. 5D is a graph of the comparative analysis of the performance of SIENNA and SCENIC, the state-of-the-art model, in the binary classification task of clinical data.

FIG. 6A is a series of MRI images of “patient 3” with known GBM, subjected to preprocessing, and the corresponding output of the SIENNA model for classification of tumor types.

FIG. 6B is a series of MRI images of “patient 1” with known MET, subjected to preprocessing, and the corresponding output of the SIENNA model for classification of tumor types.

FIG. 6C is a table that presents the results of SIENNA's patient-wise analysis, showing the F1 score along with the values of true positive (TP), true negative (TN), false positive (FP), and false negative (FN) for each input.

### DETAILED DESCRIPTION OF THE INVENTION

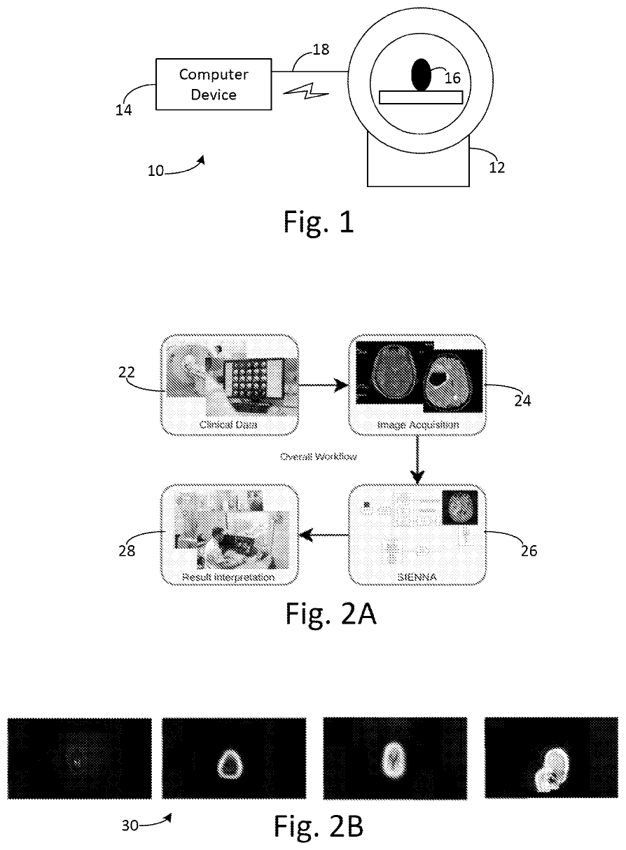

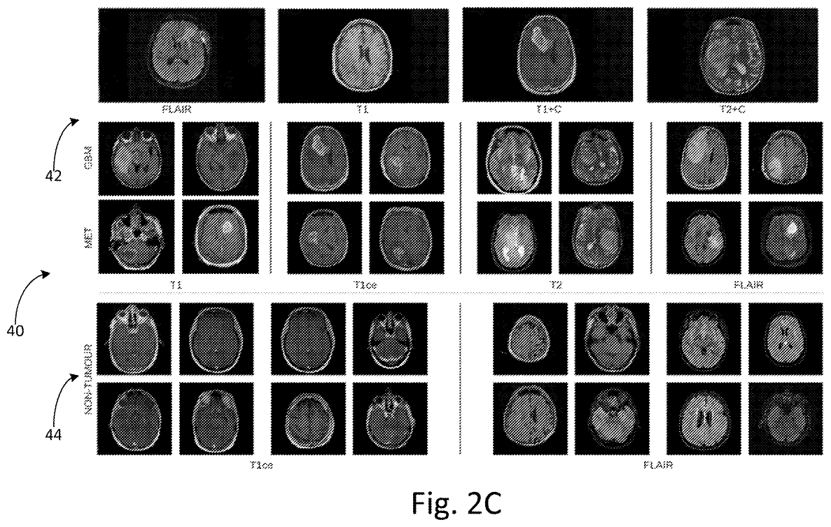

Referring now to the drawings in which like numbers represent like elements throughout the several views, FIG. 1 is a diagram of one embodiment of the present system 10 utilizing an MRI machine 12 as the scanning device to provide diagnostic images of a human brain, such as images shown in FIG. 2B-2C. In this embodiment, the system 10 for determining the presence of cancerous tumors within diagnostic images of a human body includes a scanning device, here MRI 12 generating one or more diagnostic images of a human body, which here are brain images from the head 16 or a patient, such as the images shown in FIGS. 2B-2C. The scanning device (MRI 12) selectively transmits the one or more diagnostic images to other computer devices, such as computer device 14. Computer device 14 can be one or more physical computer platforms, or could be virtual drives located in the Cloud. And the transmission can be from a hardwired connection 18 or through a network and wireless connection.

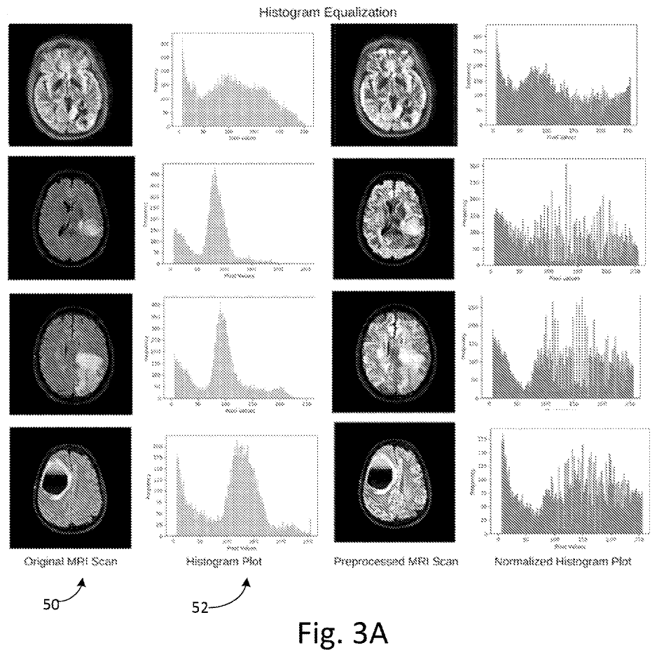

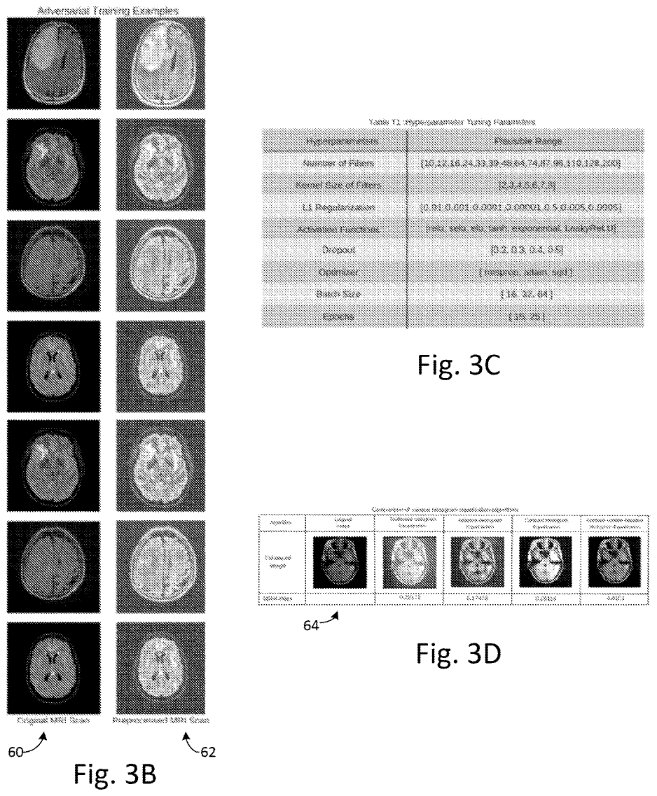

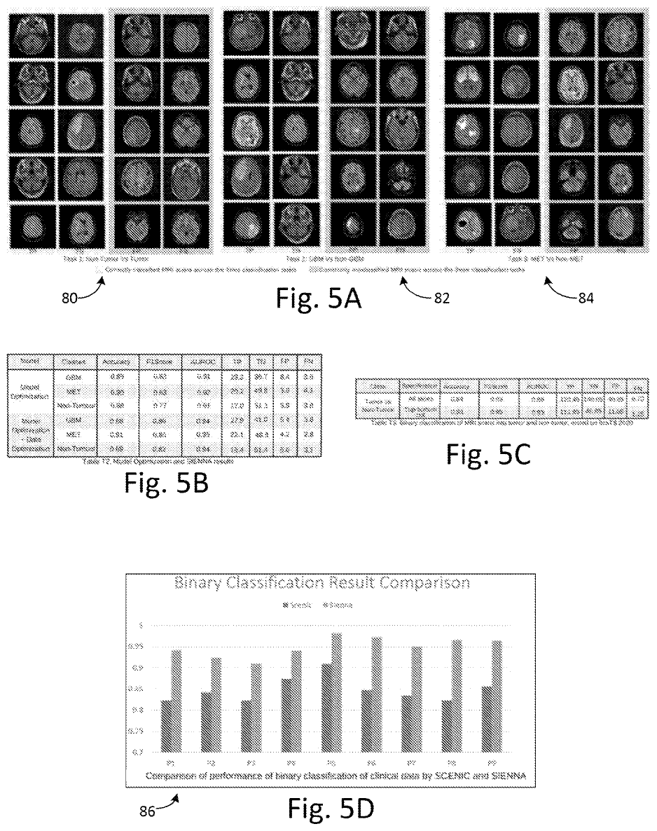

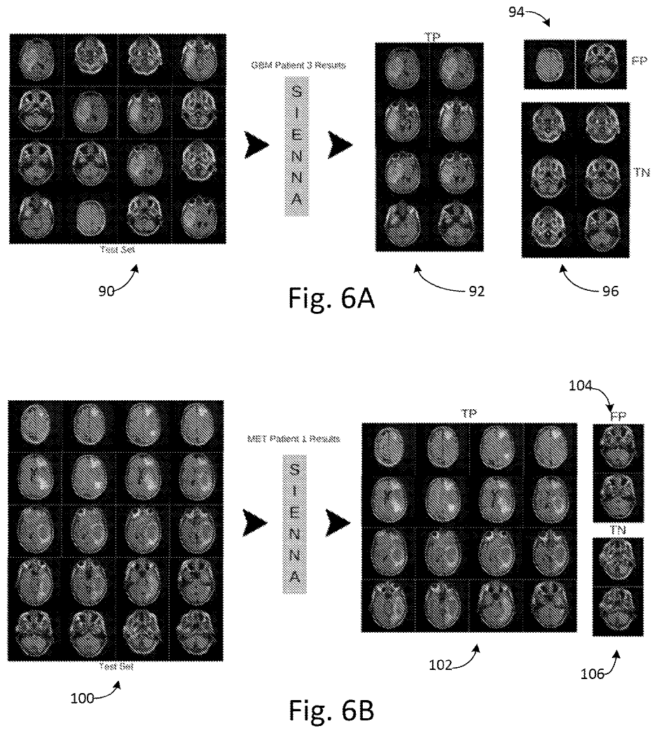

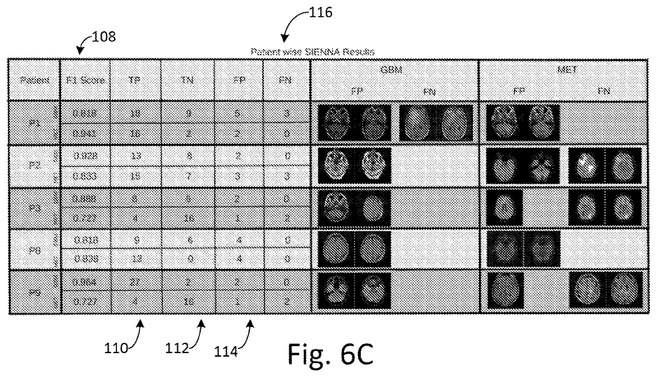

The computer device 14 is then configured, via programming in software, middleware, firmware, or a combination thereof, to perform the steps of receiving the one or more diagnostic images from the scanning device (MRI 12), creating a plurality of tiles for each received image, as further described below, and then computing a histogram for each tile, as shown in FIG. 3A. The computer device 14 then, as further detailed below, distributes an intensity for each tile, creating a cumulative distribution function scaling for each tile, conducts a bilinear interpolation to thereby stitch together the processed tiles, and then combines the processed tiles into in a final equalized image, analyzing the final equalized image to determine a presence of cancerous tumors within the diagnostic image, as shown in FIGS. 5A-6C. In one embodiment, the analyzing the final equalized image is analyzing the final equalized image with a convolutional neural network. Further, the computer device 14 can be further configured to perform the step of training a predictive model based upon the analyzing of the final equalized images. Additionally, the computer device 14 can further be configured to perform the steps of producing a training data based upon the received one or more diagnostic images, introducing adversarial perturbations in the training data to train the predictive model.

When the one or more diagnostic images are MRI images, such as in FIGS. 2B-2C, the computing of a histogram is computing a histogram equalization, as shown in FIG. 3A. And as embodied here, the diagnostic images are one or more images of a human brain, but other images and scanning techniques can likewise be used in the present invention for medical diagnoses, as would be apparent to one of skill in the art.

Further, as embodied herein, the analyzing the final equalized image to determine a presence of cancerous tumors within the diagnostic images can be determining a presence of glioblastomas, a further described below. As also described herein, the training of the predictive model can include tuning of hyper-parameters from the group of a weight penalty factor, learning rate, and momentum.

One constructed embodiment of the present system 10 is called SIENNA, a deep learning platform developed with access to a non-processed de-identified clinical DICOM dataset of 386 scans, comprising 153 GBM, 125 MET clinically identified brain tumors, and 108 non-tumor scans. SIENNA system addresses key challenges and strategies in working with clinical data and employ cutting-edge algorithms and tools to guide optimizations. This includes Synthetic Minority Over-Sampling Technique (SMOTE) to address class imbalance, Hyperas for optimizing hyper-parameters, histogram equalization for scan standardization, and adversarial training to ensure model robustness against different types of adversarial attacks. Capitalizing on the distinctive attributes of the convolutional neural network (CNN) architecture and its capacity to extract intricate hierarchical features from data, our clinical data optimized SIENNA model exhibits robust tissue pathology typing, demonstrated here by discrimination of GBM versus MET tumor pathologies. The versatility of SIENNA in handling both raw and processed clinical data renders it an indispensable resource for enhancing clinical treatment workflow speed and efficacy to promote superior patient outcomes.

FIG. 2A is an overview and representation of MRI clinical dataset process flow, illustrating the clinical workflow from clinical data intake 22, to MRI image scans 24, to SIENNA processing 26, to output interpretation 28 by neurosurgeons, including collection, preprocessing, input, and output interpretation of data. FIG. 2B is a series of images 30 are examples of de-identified raw MRI scans deemed noisy and unfit for training. FIG. 2C is a series of images 40 of MRI scans across different modalities present in the clinical dataset are shown along with representative images from GBM and MET that are either tumor (top panel 42) or non-tumor (bottom panel 44).

The retention of data features during pre-processing of DICOM images. SIENNA is designed to fit seamlessly into the clinical workflow with DICOM images (FIGS. 2A-2B) and to achieve greatest retention of raw features for distinguishing GBM versus MET tumor neoplasms (FIGS. 2B-2C). Particular challenges intrinsic to working with clinical datasets were anticipated but avoided through common extensive preprocessing techniques such as re-orientation to LPS/RAI, N4 Bias correction, and Skull-stripping, which are seen with publicly available data. Clinical data evaluation includes addressing factors of data de-identification and clinical expert verification as well as natural patient and tumor variations contributing to noise that must be minimized, data annotation, and metadata creation. Here we evaluated de-identified clinical data obtained through an IRB-approved collaboration with Albany Medical College and structured in a patient-centric manner, with GBM and MET patient folders comprising multiple imaging modalities including T1, T1-contrast-enhanced (T1-ce), T2-weighted (T2), and fluid-attenuated inversion recovery (FLAIR). Data from 20 patients is evaluated, 10 belonging to each of the GBM and MET categories.

Initial modality screening revealed value of T2 and FLAIR modalities for highest accuracy, consistent with their preferred use in MR diagnostics clinically. Given the high dimensionality of FLAIR and its ability to effectively distinguish tumor physiology, this modality was selected for all subsequent analyses. To facilitate effective training, validation, and testing of the SIENNA deep learning model, we segregated each patient Z-stack of horizontal MRI scans into tumoral and non-tumoral classes. Horizontal images that contained incomplete tissue data (uppermost images, primarily skull area) or heightened data complexity (lowermost images, primarily non-brain tissues) were removed (FIG. 2B) to reduce noise from limitations of a small dataset. All MRI scans were annotated with a uniform standardized naming format across the 20 patients to enable ease of analysis and verification during the testing phase. Shown are MRI scans across different MRI modalities (FIG. 2C), including GBM and MET tumors and non-tumor samples across modalities.

The variable sizes of brain tumors in patient datasets impacts the number of non-tumor data images within a given patient horizontal Z-stack. When the tumor is large, the tumor class constitutes the majority of the horizontal slices in a patient dataset and the non-tumor scan becomes a minority class. This class imbalance can lead to biased models that favor the majority class and fail to learn the patterns and features of the minority class adequately. To address this issue, data augmentation techniques can be employed to restore class balance within and across patient datasets. The Synthetic Minority Over-Sampling Technique (SMOTE) is a popular method for minority class oversampling, which involves generating synthetic samples by interpolating between minority class examples. By replicating samples using k-nearest neighbors of the low-frequency class, the SMOTE technique forces the network to allocate resources to the analysis of the minority class, thereby improving model performance.

FIGS. 3A-3D show a model optimization for stability and robustness using histogram equalization, Adversarial Training and Hyperas. These are applied selected techniques for enhancing stability and robustness of deep learning models, such as histogram equalization and adversarial training that are presented here alongside their respective evaluation metrics and results. FIG. 3A is a series of images 50 and graphs 52 illustrating histogram equalization, which showcases improved contrast equalization of the images in the dataset, represented along with histograms of original and enhanced scan. FIG. 3B is a series of MRI images illustrating original MRI scans 60 and visualizing some adversarial examples 62 used in the training process.

The variability in MRI scan intensities and quality across patients due to patient movement, technical characteristics of each MRI system, acquisition parameters and scanning conditions can create incomparability in datasets. When each of the scans have varying pixel intensities, the features the deep learning model learns will vary as well. We applied histogram equalization to address this issue by normalizing scan intensity and reducing sensitivity to illumination and other factors that can impact feature visibility (FIGS. 2A, 2D). This facilitated better differentiation and recognition of distinct brain features by the model.

The present system allows augmentation of SIENNA by parameter and hyper-parameter optimization and adversarial training. SIENNA employs a multiple CNN framework to perform feature engineering and extraction of complex nonlinear dependencies from input images mapped to output classes. CNNs are the AI method of choice for complex medical data sets to bring greatest flexibility in handling a broad range of parameters and hyper-parameters critical for data optimization and analysis. While parameters like weight and bias are learned during training, hyper-parameters such as weight penalty factor, learning rate, and momentum are tuned features. Model performance requires trial and error experimentation of different parameter combinations while retaining greatest generalizability.

FIG. 3C is a table of Hyperas describing the various parameters and their respective plausible ranges of values provided for hyperparameter tuning. We applied the HYPERAS algorithm to maximize performance on new data. The algorithm serves as a convenient interface for the Hyperopt library and automates the search for optimal hyper-parameters by exploring a range of user-defined search spaces. Various modeling strategies were applied to the data to estimate the achievable level of test error performance, with recall serving as the performance metric to be optimized by HYPERAS (FIG. 2C). In medical settings AI diagnostics may generate patient critical decisions in which avoiding misdiagnosis is essential. Image classification accuracy can be defined by metrics from the confusion matrix, such as true positive (TP), true negative (TN), false positive (FP), and false negative (FN) image analysis. To measure the discrepancy between the predicted and true values of the outputs by SIENNA, the loss function categorical cross-entropy, along with the false positive and false negative metrics, were used as the objective function during the optimization process. False positives and false negatives quantify misclassified scans and can be passed as loss functions to optimize the model towards decreased gradient.

FIG. 3D is a chart 64 of the comparison of histogram equalization algorithms—Visualization of different histogram equalization algorithms tested and evaluated using the SSIM index. CNN models offer a versatile set of functions to optimize but require avoidance of common deep learning issues of overfitting and outlier sensitivity in neural networks. To address this problem, adversarial training was utilized to introduce ‘adversarial examples’ as training samples, which are slightly modified to intentionally cause misclassification by machine learning classifiers. The augmentation process greatly enhanced SIENNA ability to distinguish between pertinent and irrelevant features, thereby improving the model's overall performance (FIG. 2B). However, it should be noted that noise introduced into the training data must be calibrated with careful consideration, as excessive noise has the potential to undermine the model's ability to identify meaningful patterns and attributes. An essential balance between the amount and nature of the noise introduced into the training dataset, gained by familiarity with the data features, helps to ensure that the model achieves optimal performance.

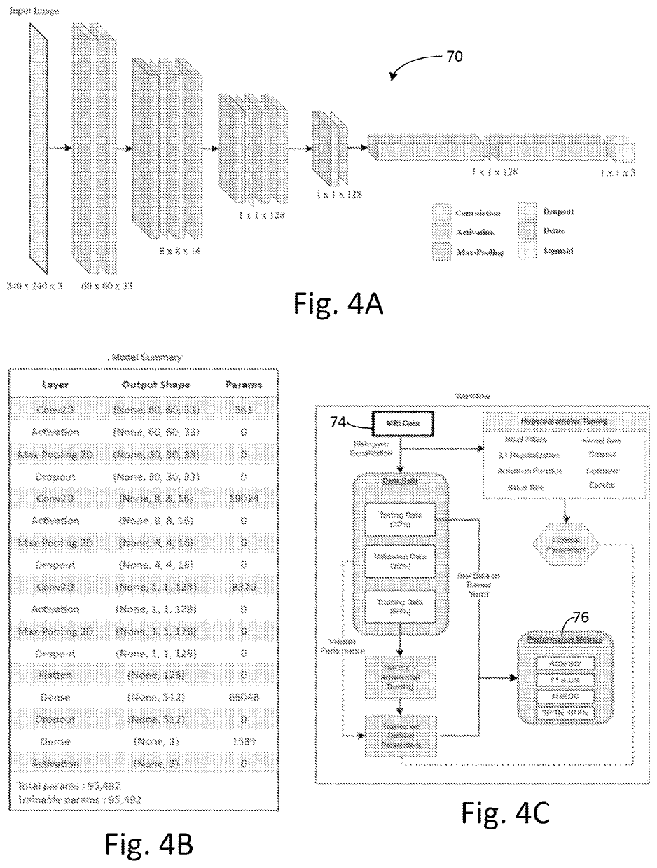

The SIENNA CNN architecture and data processing pipeline are shown in FIGS. 4A-4C. The workflow chart of the SIENNA model indicates data optimization and model optimization at different stages of development, leading to the final optimized SIENNA model. FIG. 4A. illustrates a multilayer CNN 70, consisting of several convolutional and pooling layers, followed by fully connected layers and a final output layer. The depicted layers in the architecture possess both spatial and depth dimensions, which serve to illustrate the data shape and its transformation as it passes through the network.

FIG. 4B is a table summarizing the layers and output of the CNN architecture—includes the number of layers, output shape, and the number of parameters. It is also shown that SIENNA excels at binary and multiclass pathology classification tasks on diverse datasets. An assessment of SIENNA's performance in both binary and multiclass classification tasks. A non-interdependent multi-classification analysis can be used to reduce misdiagnosis risk. The subtasks classify input MRI scans into one of three categories, namely Non-Tumor, Glioblastoma (GBM), or Metastatic (MET). The positive classes for each subtask in the SIENNA model serve to emphasize the distinctive characteristics of each type of tumor, thereby enabling the model to discern between them. The final classification label of the input MRI scan is obtained using a comparative analysis of the results, for each subtask. This represents the SIENNA decision output for classification of the MRI scan as GBM or MET. Our evaluation of SIENNA's performance involves the use of various performance metrics such as accuracy, F1 score, Area Under the Receiver Operating Characteristic Curve (AUROC), as well as the number of False Positives (FP) and False Negatives (FN) [29]. The MR images for True Positive (TP), True Negative (TN) and FP and FN outcomes (FIG. 5A) and assessments (FIG. 5B) reveal the crucial role of an integration-based strategy in the development and optimization of deep learning models for complex medical imaging tasks. Furthermore, our 3-label multiclass classification model is more efficient and provides more detailed and nuanced predictions compared to a hierarchical 2-binary classification stacked model.

FIG. 4C is a diagram illustrating the SIENNA data processing pipelines. To further evaluate the performance of SIENNA, the model was tested on the highly pre-processed BraTS 2020 dataset of MRI data 74, which is a clinically acquired dataset of lower and higher grade GBMs. The BraTS dataset has been optimized to provide high-quality medical imaging data by using intensive preprocessing techniques like intensity normalization, bias field correction, and noise reduction. The dataset consists of NIfTI files divided into four different MRI modalities, each containing 155 sliced images of dimension 240×240×3. Although this dataset is limited to the tumor type Glioma, it serves as a standardized resource for the development and evaluation of computer-aided diagnosis (CAD) systems for brain tumor segmentation. SIENNA was able to classify tumors accurately (performance metrics 76) on a subset of the BraTS 2020 dataset consisting of 20 patients with a F1 score of 97% and 96% Accuracy (FIGS. 5C-5D). One of the strengths of SIENNA is its versatility in accommodating both clinical and extensively pre-processed data, which makes it a valuable and adaptable tool for different medical applications.

FIGS. 5A-5D illustrate a collective data analysis of SIENNA's high accuracy rates in discriminating GBM and MET tumors. The figure illustrates the stratification of MRI scans into distinct categories and presents the evaluation metrics for the SIENNA model across various tasks, including classification accuracy for Task 1 (Tumor vs Non Tumor), Task 2 (GBM vs Non-GBM), and Task 3 (MET vs Non-MET) averaging an accuracy range of 88-91%.

FIG. 5A is a series of MRI images highlighting the most frequently correctly and incorrectly classified samples, with the top 20 MRI images that were classified with the highest and lowest accuracy for each task are shown. Specifically, a non-tumor versus tumor images group 80, a glioblastoma version non-glioblastoma group 82, and a metastasis versus not non-metastasis group 84 are illustrated.

FIG. 5B is a table of the performance evaluation on clinical data that represents the evaluation metrics estimated as an average of 100 runs of SIENNA. FIG. 5C is a table of the performance evaluation of SIENNA for binary classification tasks on the extensively preprocessed BraTS 2020 dataset. FIG. 5D is a graph 86 of the comparative analysis of the performance of SIENNA and SCENIC, the state-of-the-art model, in the binary classification task of clinical data. The graph 86 shows accuracy in on a binary 0-1 scale.

FIGS. 6A-6C illustrates a patient-wise performance evaluation of SIENNA. FIG. 6A is a series of MRI images of “patient 3” with known GBM, subjected to preprocessing, and the corresponding output of the SIENNA model for classification of tumor types. The test set 90 is interpreted by SIENNA and a true positive set 92 is created, along with a false positive set 94 and a true negative set 96.

FIG. 6B is a series of MRI images of “patient 1” with known MET, subjected to preprocessing, and the corresponding output of the SIENNA model for classification of tumor types. The test set 100 is interpreted by SIENNA and a true positive set 102 is created, along with a false positive set 104 and a true negative set 106.

FIG. 6C is a table that presents the results of SIENNA's patient-wise analysis (P1, P2, P3, P8, P9), showing the F1 score 108, along with the values of true positive (TP) 110, true negative (TN) 112, false positive (FP) 114, and false negative (FN) 116 for each input. The MRI images of the groups are then displayed.

Accordingly, SIENNA evaluates patient-specific GBM or MET tumor. A personalized assessment of the model's performance on individual patient cases, typical in clinical practice, was performed for FIGS. 6A-6C. Detailed patient-wise evaluation provides an opportunity to identify previously unrecognized patterns or relationships within the data, leading to new insights in the field of brain tumor diagnosis and treatment. The efficacy of the model in precise classification of brain tumors with minimal false positives and negatives was exhibited through the patient-wise processing of input data for GBM-3 (FIG. 6A) and MET-1 (FIG. 6B) patient cases and evaluation of performance metrics (FIG. 6C). In summary, the development and optimization of SIENNA as a multiclass classification model for diagnostic distinction of clinical MRI scans as GBM and MET tumors demonstrates deep learning efficiency and high accuracy for brain tumor classification.

For SIENNA performance-enhancing design strategies and optimization techniques, model training, validation and testing. The fine-tuning of a neural network model for a specific problem statement and diverse data complexities relies on the optimal selection of hyper-parameters and model parameters, learned through training and updated via backpropagation. Parameters can be defined as internal configurations that determine the model's functionality and must be fine-tuned to create a robust and reliable deep learning platform. The training process is important in this aspect as it is the set from which the neural network is able to extract features to distinguish classes. Convolution is an efficient technique for feature extraction, reducing data dimensionality and generating feature maps using kernels as feature identifiers. However, data diversity and richness in the training set are crucial for optimal feature extraction. Unfortunately, there are no established criteria for distinguishing information-rich scans from clinical datasets, which are often provided in a patient-wise format of successive z-slice brain stacks. To address this, shuffling the datasets randomly can be helpful. During training, it is important to avoid presenting the model with successive scans belonging to the same class, as these are likely to contain similar information.

A large error between the target and network output can indicate new information in the data and represents the kind of data the model has not been trained upon. In cases where a certain MRI type presents unique complexities, images that are frequently misclassified can be exclusively added to the training set. In the context of deep learning, evaluating the performance of a model solely on the training set is insufficient as it only reflects its ability to memorize the training data, thereby limiting its ability to generalize to unseen data. This necessitates the use of a validation set, which measures the model's accuracy on an independent dataset during training, allowing it to learn and adapt to new features in the data distribution. This process is crucial for ensuring that the model does not overfit the training data and improves its performance on unseen data.

In one embodiment, the present system 10 utilizes convolutional neural network (CNN) formulations. In the context of a conventional multi-layer neural network, consider wₙ and bₙ to be the weight and bias parameters learned through the training process, and Tₙ as the activation function of the network which produces the final output. The output of this model can be represented as yₙ=Tₙ([wₙ,bₙ]*xₙ), where xₙ is the input to the n^{th} layer. The output of one layer in a multi-layer neural network is used as the input for the subsequent layer until a final class prediction is made. Weights and bias of the model will be constantly updated with the aim of minimizing the average training cost Cₜᵣₐᵢₙ over the training set {x₁, x₂ . . . xₙ}, using gradient based learning methodologies. The selection of model architecture and loss function can be deemed critical in enhancing the generalization performance, thereby being reliant on the rapidity and accuracy of gradient descent optimization.

Given the multi-class nature of our model, the categorical cross entropy was selected as the primary loss function for estimating errors. In addition, we employed the use of false positive and false negative metrics as auxiliary loss functions. This is particularly relevant in the field of medical diagnosis, where the implications of FP and FN errors can be significant, and the ability to optimize the model with respect to these metrics can be of critical importance. The total cost function can be represented as

### Where

$${C}_{train} = {C}_{CE ( predicted , true )}^{} + {FP}_{( predicted = tumor , true = non - tumour )} + {FN}_{( predicted = non - tumor , true = tumor )}$$

$${C}_{CE ( predicted , true )}^{} =$$

The gradient refers to the vector of partial derivatives of the loss function with respect to each learnable parameter of a neural network. In the process of optimizing the network's performance, an update to the learnable parameters is performed through a single step along the gradient direction, which is aimed at minimizing the loss function. This step can be mathematically represented as

$$w = w - γ \frac{\partial C}{\partial w}$$

Where γ, a scalar constant, is the learning rate that controls the tradeoff between the speed and stability of weight convergence. It determines the degree to which the weight parameters are updated at each iteration of the training process.

In practice, the gradients of error functions are computed on batches of data, and stochastic gradient descent (SGD) is a popular algorithm that can be used for this purpose. However, for our purposes, we have adopted the Adam optimization algorithm, which is a more sophisticated version of gradient descent and is available in the Python programming language. Medical MRI datasets exhibit a high degree of variability in data distribution over time, and a deep learning model must be able to adapt to these constantly changing functions to achieve generalizability and avoid introducing noise into the data analysis process. Stochastic gradient descent is a suitable algorithm for problem statements where data distribution changes over time.

The present system 10 can also utilize adaptive histogram equalization techniques for enhancement of contrast across MRI data. The standardization of MRI scans is critical for accurate analysis and diagnosis, as variations in pixel intensities can result from various factors such as differences in scanning parameters and anatomical variations. These variations can make it difficult to compare scans between patients and may lead to errors in interpretation. Histogram equalization can mitigate these variations by normalizing MRI scans and improving comparability among patients. This technique equalizes the histograms, reducing the variations in intensity and enabling a more standardized comparison of scans between patients. Research has demonstrated that histogram equalization can be effective in improving the accuracy and reliability of MRI analysis, particularly in clinical settings where consistent and accurate diagnosis is critical and is applied here.

Consider an jpeg MRI image x(p,k), where p denotes the position coordinates and k denotes the color channel. It should be noted that MRI images are typically grayscale, but the pixel intensity range may vary due to differences in machine software or other parameters. Therefore, we represented the possible intensities of the MRI images as the range [G₀−(G_{L}−1)], where G, is the minimum intensity value and G_{L}−1 is the maximum intensity value. The probability density function (PDF) of an intensity level i can be expressed as

PDF x(i)=nᵢ/n where G₀≤i≤(G_{L}−1)

Where nᵢ represents the total number of pixels with intensity value i, and n represents the total number of pixels in the MRI image.

The cumulative probability function (CDF) of the corresponding PDF can be defined as: (written in the context of this problem; it is a generic expression.)

$$CDF x ( i ) = {\sum }_{G}^{G - 1} PDF x ( i )$$

The transformation function T maps the cumulative distribution function of the MRI scan to a uniform distribution as:

$$T = {G}_{0} + ( ( {G}_{L} - 1 ) - {G}_{{0}_{}} ) \times CDF x ( i )$$

$$y = \{ T \times ( p , k ) \vert ∨ \times ( p , k ) ϵ G \}$$

While histogram equalization can be effective in improving the contrast and visibility of an image it may not always lead to perceptually better results. Therefore, evaluating the quality of these images after histogram equalization is needed. In our study, we utilized the Structural Similarity Index (SSIM) to evaluate the structural similarity between the original MRI scans and the enhanced scans and represents the quantification of image structure, contrast, and luminance through the respective formulations mentioned below. Here, x(p,k) and y(i,j) denote the two scans being compared.

The structure component computes the correlation between the two scans and can be defined as:

$$structure ( x , y ) = \frac{{σ}_{xy} + c}{{σ}_{x} {σ}_{y} + c}$$

(these notations not defined)

The contrast component measures the disparity in contrast between the two scans and can be defined as:

$$contrast ( x , y ) = \frac{2 {σ}_{x} {σ}_{y} + c}{{σ}_{x}^{2} + {σ}_{y}^{2} + c}$$

The luminance component calculates the variation in brightness between the two MRI scans and can be defined as:

$$luminance ( x , y ) = \frac{2 {μ}_{x} {μ}_{y} + c}{{μ}_{x}^{2} + {μ}_{y}^{2} + c}$$

Where cₙ=(KₙG)²: included to avoid instability K is a constant parameter that is typically set to a value less than 1, and G is the maximum possible pixel value in the scan.

Together, these three components allow for a comprehensive analysis of the structural similarity between two scans, using the equation:

$$SSIM ( x , y ) = {[ l ( x , y ) ]}^{α} \cdot {[ c ( x , y ) ]}^{β} \cdot {[ s ( x , y ) ]}^{γ} ,$$

where l(x,y), c(x,y), and s(x,y) represent the luminance, contrast, and structure components, respectively, and α, β, and γ are weighting parameters that determine the relative importance of each component.

The equation can be rewritten as:

$$SSIM ( x , y ) = \frac{( 2 {μ}_{x} {μ}_{y} + {c}_{1} ) ( 2 {σ}_{xy} + {c}_{2} )}{( {μ}_{x}^{2} + {μ}_{y}^{2} + {c}_{1} ) ( {σ}_{x}^{2} + {σ}_{y}^{2} + {c}_{2} )}$$

Here, μx and μy are the means of the two scans being compared, and σₓ², σ_{y}², and σ_{xy}^{□} are the variances and covariance of the two scans.

Effective intensity equalization of medical images is hypothesized to result in a more uniform histogram of the equalized image, thereby increasing its similarity with a reference image that has a uniform histogram. This is expected to be reflected in a higher SSIM value calculated between the equalized and reference images, indicating a greater degree of structural similarity between the two images. Our study, as depicted in FIG. 3D, compared different histogram equalization techniques including conventional histogram equalization, adaptive histogram equalization, and their respective modifications. Our findings suggest that the Contrast Limited Adaptive Histogram Equalization (CLAHE) algorithm outperformed other techniques in enhancing contrast and highlighting anatomical structures in brain scan images. It effectively minimizes the over-amplification of noise in homogeneous regions of an image, something other equalization algorithms failed to do. This highlights the effectiveness of CLAHE as an optimal intensity normalization algorithm for MRI scans enhancement. The CLAHE algorithm partitions the scan into non-overlapping tiles of a specified size, in this case 8×8 pixels, resulting in 900 tiles. Smaller tile sizes are preferred for finer detail. Histogram equalization is performed for each tile, with a contrast limit applied to prevent over-amplification of noise in homogeneous regions. This contrast limit can be formulated as (these above sentences are generic ones).:

clip limit=minClipLimit+N(CLIPS)×(N(AVG)−minClipLimit)

Where N (CLIPS) is the total number of clips, N (AVG) represents the average number of pixels in the mapped image and minClipLimit represents the minimum allowable clip limit. This value is typically set to a small positive number, such as 0.01, to ensure that some amount of contrast amplification occurs for all tiles. The clip limit formula is used to determine the maximum allowable contrast amplification for each tile, based on the average number of pixels in the mapped image and the specified minimum clip limit. This helps to ensure that the contrast amplification is optimal for each tile, without introducing excessive noise.

In one embodiment, the method involves the computation of histogram, intensity redistribution, and cumulative distribution function scaling for each tile of the image, followed by bilinear interpolation to stitch together the processed tiles, resulting in a final equalized image. The effectiveness of the method is evaluated through visual comparison between the original and enhanced images, along with their corresponding histogram plots, as depicted in FIG. 3A. The histogram equalization process is demonstrated to significantly improve image quality, as evidenced by the changes in image intensity distribution.

Overfitting is a well-known obstacle in deep learning when training models with limited data. Adversarial training has emerged as a promising strategy to address this challenge, by leveraging adversarial examples to improve the model's robustness and generalization performance. This method introduces adversarial perturbations in the training data, which enables the model to handle small variations in input and perform effectively with new data. We use the variable h_{θ} to represent the neural network model, \hat{x} to represent the generated adversarial example and y to represent the true label. The goal is to generate adversarial examples that maximize the loss function to help improve the model's performance. Therefore, the optimization problem can be represented as a maximization of loss:

F(x)=argmax L(y,h_{θ}\hat{x})

Here, L represents the loss function, which computes the difference between the predicted output h(,x) and the true label y.

Let the perturbation function be:

δ_{∞}={δ:∥δ|≤∞}

To ensure that \hat{x} is close to our original input x, we optimize over the perturbation δ to x, and then by optimizing over δ:

F(x)=argmax L(y,h_{θ}\hat{x}+δ) where δ≤ε

We are optimizing over the perturbed input \hat{x}+δ, which is expected to be closer to the original input x than a completely adversarial example would be. By doing so, we are able to generate adversarial examples that are both effective in improving the model's performance and close to the original input x.

For, the Fast Gradient Sign method (FGSM) was utilized to create adversarial examples, where a gradient calculated using a loss function is added as perturbations to the input data.

grad(x)=∇L(y,h_{θ}x+δ)

FGSM generates adversarial examples by computing the gradient of the loss function with respect to a perturbation δ via backpropagation. By applying gradient descent to δ, we can obtain the direction of perturbation that maximizes the difference between the model's prediction and the desired output. This approach is based on the sensitivity to variations, and the intuition that small changes to the input data can cause significant changes to the output of the model. By leveraging the gradient information of the loss function, we can perturb the input data in a way that maximizes the objective.

F(x)=argmax L(y,h_{θ}\hat{x}+[δ+grad(x)]) where δ≤ε

Once the adversarial examples have been generated, they can be incorporated into the training process by including them as input data. The model's parameters are then updated based on the loss calculated from both the original and adversarial examples. By adjusting the magnitude of perturbations, varying levels of robustness can be achieved. Although this technique improves the generalization ability of models in deep learning, a trade-off exists between robustness and accuracy, where an overly robust model may decrease accuracy on normal data. Therefore, it is crucial to balance this trade-off, and acknowledge that adversarial training may not be suitable for certain data types or models due to computational costs.

Biomedical imaging provides an invaluable resource as an integral part of patient care. MRI has a historical link to tumor pathologies but remains non-optimized analytically for clinical applications. Technology advances that increase Tesla power and provide vastly expanded detail, will only exacerbate the demands for trained medical experts to evaluate MRI data and require additional training, adding to time demands on radiologists to review and turnaround diagnostics on images. Given the increasing demand on radiologist and neurosurgeon clinicians in patient care and a high level of misdiagnosis from MRI alone that require multiple additional patient evaluations, the implementation of AI deep learning analysis of clinical data for MRI has become imperative. Currently development of cutting-edge AI platforms have focused on detection of presence or absence of tumor pathologies, such as the deep learning SCENIC algorithm developed by this group and tumor segmentation for detecting tumor edges. Here we develop SIENNA, a novel AI deep learning platform for distinguishing brain pathologies, applied here to GBM versus MET neoplasms. Our analysis reveals the challenges associated with applying extensively preprocessed clinical data for later model generalization.

SIENNA is a powerful deep learning AI algorithm trained on a small, minimally processed clinical dataset which achieves high accuracy typing (88-91%) of GBM and MET tumor pathologies, that is expected to increase in robustness with expanded data analysis, typical of AI platforms. Preceding the development of SIENNA, an analysis of post-analyzed tumors upon resection revealed high misdiagnosis (85%) by MRI alone, reflecting the variability of these tumors and challenges even for clinical experts. SIENNA works on minimally processed MRI dicom data to fit seamlessly into the clinical workflow. The development of SIENNA also points to the value of numerous tools available to optimize deep learning platforms, such as Synthetic Minority Over-Sampling Technique (SMOTE) to address class imbalance, Hyperas for optimizing hyper-parameters, histogram equalization for scan standardization, and adversarial training to ensure model robustness against different types of adversarial attacks. By capitalizing on the distinctive attributes of the convolutional neural network (CNN) architecture and its capacity to extract intricate hierarchical features from data, our clinical data optimized SIENNA model exhibits robust tissue pathology typing, demonstrated here by discrimination of GBM versus MET tumor pathologies. The versatility of SIENNA in handling both raw and processed clinical data renders it an indispensable resource for enhancing clinical treatment workflow speed and efficacy to promote superior patient outcomes.

SIENNA's ability to handle raw clinical data with minimal preprocessing, along with publicly available data, sets it apart from other state-of-the-art methods. Furthermore, SIENNA shows potential for future adaptation and application to a range of brain physiology and pathology tasks in clinical biomedical imaging. To improve the model's performance and robustness, analysis on larger clinical datasets, including MRI data from different manufacturers, will be necessary. Furthermore, the integration of AI-based pre-diagnostic predictions, classification of additional brain tumor types and stages, and comorbidity analysis can be incorporated to enhance the model's utility. SIENNA's AI capabilities can continue to learn from complex and diverse clinical MRI datasets brings MRI robustness in biomedical neurosurgical treatment pre-planning. Moreover, the versatility of SIENNA's AI framework allows for its potential application in other brain pathologies and expanded use in artificial generalizable intelligence.

Also attached hereto as Appendix A is source code to enable one embodiment of the present invention that can be executed on computer device 14.

The corresponding structures, materials, acts, and equivalents of all means or step plus function elements in the claims below, if any, are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of one or more aspects of the invention and the practical application, and to enable others of ordinary skill in the art to understand one or more aspects of the invention for various embodiments with various modifications as are suited to the particular use contemplated.

What is claimed is:

1. A system for determining the presence of cancerous tumors within diagnostic images of a human body, comprising:
a scanning device generating one or more diagnostic images of a human body, the scanning device selectively transmitting the one or more diagnostic images to other computer devices; and
a computer device configured to perform the steps of:
receiving the one or more diagnostic images from the scanning device;
creating a plurality of tiles for each received image;
computing a histogram for each tile;
redistributing an intensity for each tile;
creating a cumulative distribution function scaling for each tile;
conducting a bilinear interpolation to thereby stitch together processed tiles;
combining the processed tiles into in a final equalized image; and
analyzing the final equalized image to determine a presence of cancerous tumors within the diagnostic images.

2. The system of claim 1, wherein the analyzing the final equalized image is analyzing the final equalized image with a convolutional neural network.

3. The system of claim 1, wherein the computer device further configured to perform the step of training a predictive model based upon the analyzing of the final equalized images.

4. The system of claim 3, wherein the computer device further configured to perform the steps of:
producing a training data based upon the received one or more diagnostic images; and
introducing adversarial perturbations in the training data to train the predictive model.

5. The system of claim 1, wherein:
wherein the one or more diagnostic images are MRI images; and
computing a histogram is computing a histogram equalization.

6. The system of claim 5, wherein the diagnostic images are one or more images of a human brain.

7. The system of claim 6, analyzing the final equalized image to determine a presence of cancerous tumors within the diagnostic images is determining a presence of glioblastomas.

8. The system of claim 4, wherein the training of the predictive model includes tuning of hyper-parameters from the group of: weight penalty factor, learning rate, and momentum.

9. A computer-implemented method for determining the presence of cancerous tumors within diagnostic images of a human body, comprising:
generating one or more diagnostic images of a human body at a scanning device;
selectively transmitting the one or more diagnostic images from the scanning device to other computer devices;
receiving the one or more diagnostic images from the scanning device;
creating a plurality of tiles for each received image;
computing a histogram for each tile;
redistributing an intensity for each tile;
creating a cumulative distribution function scaling for each tile;
conducting a bilinear interpolation to thereby stitch together processed tiles;
combining the processed tiles into in a final equalized image; and
analyzing the final equalized image to determine a presence of cancerous tumors within the diagnostic images.

10. The method of claim 9, wherein analyzing the final equalized image is analyzing the final equalized image with a convolutional neural network.

11. The method of claim 9, further including training a predictive model based upon the analyzing of the final equalized images.

12. The method of claim 11, further including:
producing a training data based upon the received one or more diagnostic images; and
introducing adversarial perturbations in the training data to train the predictive model.

13. The method of claim 9, wherein:
generating one or more diagnostic images is generating one or more diagnostic MRI images; and
computing a histogram is computing a histogram equalization.

14. The method of claim 13, wherein generating one or more diagnostic MRI images is generating one or more diagnostic MRI images of a human brain.

15. The method of claim 11, analyzing the final equalized image to determine a presence of cancerous tumors within the diagnostic images is determining a presence of glioblastomas.

16. The method of claim 12, wherein the training of the predictive model further includes tuning of hyper-parameters from the group of: weight penalty factor, learning rate, and momentum.

17. A system for determining the presence of cancerous tumors within diagnostic images of a human body, comprising:
a computer device configured to perform the steps of:
receiving one or more diagnostic images from a scanning device a generating one or more diagnostic images of a human body, the scanning device selectively transmitting the one or more diagnostic images to other computer devices;
creating a plurality of tiles for each received image;
computing a histogram for each tile;
redistributing an intensity for each tile;
creating a cumulative distribution function scaling for each tile;
conducting a bilinear interpolation to thereby stitch together processed tiles;
combining the processed tiles into in a final equalized image; and
analyzing the final equalized image to determine a presence of cancerous tumors within the diagnostic images.

18. The system of claim 17, wherein the analyzing the final equalized image is analyzing the final equalized image with a convolutional neural network.

19. The system of claim 17, wherein the computer device further configured to perform the step of training a predictive model based upon the analyzing of the final equalized images.

20. The system of claim 19, wherein the computer device further configured to perform the steps of:
producing a training data based upon the received one or more diagnostic images; and
introducing adversarial perturbations in the training data to train the predictive model.
